SEQUENCE LISTING (16 sequences)

SEQ ID NO: 1 - DNA, 27 bp, synthetic construct
  source 1..27: mol_type = other DNA; note = A01-T
  modified_base 1..25: mod_base = m5c
  modified_base 26: mod_base = s2c
ntnangangntntnngtntanaannnt

SEQ ID NO: 2 - DNA, 30 bp, synthetic construct
  source 1..30: mol_type = other DNA; note = A01-B
  modified_base 1..30: mod_base = m5c
ggttgtagatnggaagagnanangtntaan

SEQ ID NO: 3 - DNA, 28 bp, synthetic construct
  source 1..28: mol_type = other DNA; note = A02-T
  modified_base 1..26: mod_base = m5c
  modified_base 27: mod_base = s2c
ntanangngntntnngatntantnannt

SEQ ID NO: 4 - DNA, 31 bp, synthetic construct
  source 1..31: mol_type = other DNA; note = A02-B
  modified_base 11..31: mod_base = m5c
gtgagtagatnggaagagnanangtntgaan

SEQ ID NO: 5 - DNA, 28 bp, synthetic construct
  source 1..28: mol_type = other DNA; note = A03-T
  modified_base 1..19: mod_base = m5c
  modified_base 27: mod_base = s2c
ntanangngntntnngatntaggatgnt

SEQ ID NO: 6 - DNA, 31 bp, synthetic construct
  source 1..31: mol_type = other DNA; note = A03-B
  modified_base 1..31: mod_base = m5c
natnntagatnggaagagnanangtntgaan

SEQ ID NO: 7 - DNA, 29 bp, synthetic construct
  source 1..29: mol_type = other DNA; note = A04-T
  modified_base 1..27: mod_base = m5c
  modified_base 28: mod_base = s2c
ntananangntnttnngatntatngannt

SEQ ID NO: 8 - DNA, 31 bp, synthetic construct
  source 1..31: mol_type = other DNA; note = A04-B
  modified_base 1..31: mod_base = m5c
gtngatagatnggaagagnanangtntgaan

SEQ ID NO: 9 - DNA, 29 bp, synthetic construct
  source 1..29: mol_type = other DNA; note = A05-T
  modified_base 1..22: mod_base = m5c
  modified_base 28: mod_base = s2c
ntanangangntnttnngtntnaagagnt

SEQ ID NO: 10 - DNA, 31 bp, synthetic construct
  source 1..31: mol_type = other DNA; note = A05-B
  modified_base 1..31: mod_base = m5c
ntnttgagatnggaagagnanangtntgaan

SEQ ID NO: 11 - DNA, 29 bp, synthetic construct
  source 1..29: mol_type = other DNA; note = A06-T
  modified_base 1..27: mod_base = m5c
  modified_base 28: mod_base = s2c
ntanangangntnttnngatntnatannt

SEQ ID NO: 12 - DNA, 29 bp, synthetic construct
  source 1..29: mol_type = other DNA; note = A06-B
  modified_base 3..29: mod_base = m5c
gtnatgagatnggaagagnananntgaan

SEQ ID NO: 13 - DNA, 51 bp, synthetic construct
  source 1..51: PCR_primers = fwd_name:R01_F,fwd_seq:aatgatacggcgaccaccgca ctctttccctacacgacgctcttccgatct,rev_name:R01_R,rev_seq:caag cagaagacggcatacgagattcgccttagtgagttcagacgtgtgctcttccgatct; mol_type = other DNA; note = upstream primer for R01
aatgatacggcgaccaccgcactctttccctacacgacgctcttccgatct

SEQ ID NO: 14 - DNA, 61 bp, synthetic construct
  source 1..61: PCR_primers = fwd_name:R01_F,fwd_seq:aatgatacggcgaccaccgca ctctttccctacacgacgctcttccgatct,rev_name:R01_R,rev_seq:caag cagaagacggcatacgagattcgccttagtgagttcagacgtgtgctcttccgatct; mol_type = other DNA; note = downstream primer for R01
caagcagaagacggcatacgagattcgccttagtgagttcagacgtgtgctcttccgatct

SEQ ID NO: 15 - DNA, 54 bp, synthetic construct
  source 1..54: PCR_primers = fwd_name:R02_F,fwd_seq:aatgatacggcgaccaccgat acactctttccctacacgacgctcttccgatct,rev_name:R02_R,rev_seq:c aagcagaagacggcatacgagatctagtacggtgtcagacgtgtgctcttccgatct; mol_type = other DNA; note = upstream primer for R02
aatgatacggcgaccaccgatacactctttccctacacgacgctcttccgatct

SEQ ID NO: 16 - DNA, 58 bp, synthetic construct
  source 1..58: PCR_primers = fwd_name:R02_F,fwd_seq:aatgatacggcgaccaccgat acactctttccctacacgacgctcttccgatct,rev_name:R02_R,rev_seq:c aagcagaagacggcatacgagatctagtacggtgtcagacgtgtgctcttccgatct; mol_type = other DNA; note = downstream primer for R02
caagcagaagacggcatacgagatctagtacggtgtcagacgtgtgctcttccgatct

(19) United States
(12) Patent Application Publication
(10) Pub. No.: US 2024/0368699 A1
(43) Pub. Date: Nov. 7, 2024

(54) KIT FOR IDENTIFYING TUMOR TISSUE-OF-ORIGIN AND DATA ANALYSIS METHOD

(71) Applicant: Hangzhou ShengTing Medical Technology Ltd., Zhejiang Province (CN)

(72) Inventors: Hongcang GU, Zhejiang Province (CN); Yunfei WANG, Zhejiang Province (CN); Xianrong CHE, Zhejiang Province (CN); Xianghe MENG, Zhejiang Province (CN); Changxiao XIE, Zhejiang Province (CN); Qiongwan XIE, Zhejiang Province (CN); Wenjun WANG, Zhejiang Province (CN)

(73) Assignee: Hangzhou ShengTing Medical Technology Ltd., Zhejiang Province (CN)

(21) Appl. No.: 18/450,653

(22) Filed: Aug. 16, 2023

(30) Foreign Application Priority Data
May 4, 2023 (CN) 2023105015727

(51) Int. Cl. C12Q 1/6886; G16B 30/10; G16B 40/00

(52) U.S. Cl. CPC C12Q 1/6886; G16B 30/10; G16B 40/00; C12Q 2600/154

(57) ABSTRACT

The present disclosure provides a kit for identifying tumor tissue-of-origin, including an adapter and PCR amplification primers, where the adapter includes nucleotide sequences of A01-T, A01-B, A02-T, A02-B, A03-T, A03-B, A04-T, A04-B, A05-T, A05-B, A06-T, and A06-B; and the PCR amplification primers include nucleotide sequences of R01-F, R01-R, R02-F, and R02-R. In the present disclosure, new adapter nucleotide sequences and PCR amplification primers are designed, with higher accuracy and effectiveness. The present disclosure further provides a data analysis method for a kit for identifying tumor tissue-of-origin, including data preprocessing, alignment, methylation information statistics, quality control, and analysis. In the present disclosure, the analysis of a Beta value-based methylation index further improves a recognition ratio.

### CROSS REFERENCE TO RELATED APPLICATION

Tis patent application claims the benefit and priority of Chinese Patent Application No. 2023105015727, filed with the China National Intellectual Property Administration on May 4, 2023, the disclosure of which is incorporated by reference herein in its entirety as part of the present application.

### REFERENCE TO SEQUENCE LISTING

A computer readable XML file entitled “Sequence Listing”, that was created on Jul. 21, 2023, with a file size of 25,760 bytes, contains the sequence listing for this application, has been filed with this application, and is hereby incorporated by reference in its entirety.

### TECHNICAL FIELD

The present disclosure relates to the technical field of biomedicine, in particular to a kit for identifying tumor tissue-of-origin and a data analysis method for a kit for identifying tumor tissue-of-origin.

### BACKGROUND

Cancer metastasis refers to a process in which tumor cells fall off from a primary lesion, enter a circulatory system, transfer to other parts of the body, and continue to grow. The original tumor is called “primary lesion”, and the newly formed tumor is called “secondary lesion” or “metastatic lesion”. According to the stage of tumor cells entering the circulatory system, cancer metastasis can be divided into early dissemination model and late dissemination model. A difference between the two models lies primarily in an environment in which metastatic cancer cells mutate. In the late dissemination model, cancer cells mutate and proliferate at the primary lesion, and then metastasize through the circulatory system. At this time, the metastatic tumor has a high degree of genetic similarity with the primary tumor, and can be treated with same treatment methods as the primary tumor. In the early dissemination model, cancer cells mutate after passing through the circulatory system to reach their destination. In this model, the metastatic tumor shows a low genetic similarity with that of the cancer cells in the primary tumor, making it difficult to determine the location of the primary tumor.

When the tumor has developed into a real cancer, namely invasive cancer, it means that the cancer cells have invaded and infiltrated deeper from the place where they occurred. For invasive cancer, the metastasis advantage of cancer cells is the main cause of cancer death.

According to statistics, 90% of cancer deaths are caused by metastasis. Metastatic cancer is extremely difficult to treat. The attachment ability of cancer cells in patients is reduced or even completely lost, and the migration ability of cancer cells is enhanced, leading to the transfer of cancer cells from the original site to other body parts along with the blood or lymphatic system, thus finally forming new tumors. Tumor metastasis refers to a process in which tumor cells migrate from the original site to other body parts and continue to grow by invading the circulatory system.

Cancer of unknown primary (CUP) is a type of tumor that is diagnosed as metastatic, but has a primary location undetermined even after a comprehensive diagnostic workup. The CUP accounts for about 3% to 9% of the total number of malignant tumors, and shows a mortality rate ranking fourth or fifth among all cancers. The reason why a primary tumor cannot be determined may be that the primary tumor is too small, has been destroyed by the human immune system, or has been surgically removed. Patients with CUP generally have a poor prognosis. A median survival time after chemotherapy is 4 to 12 months, a 1-year survival rate is about 50%, and a 5-year survival rate is about 10%. Approximately 15-20% of CUP patients who receive site-specific chemotherapy have improved overall survival compared to patients treated with empiric chemotherapy.

Positron emission tomography/computed tomography (PET/CT), as once the most effective medical imaging tool to identify the primary location of CUP, was capable of diagnosing about 30% of CUP primary locations. Moller et al. detected the primary location of CUP using FDG-PET-CT. However, when the primary tumor has a small size, the PET/CT plays a limited role in tracing the origin of cancer.

Immunohistochemistry (IHC) with antibodies against tumor antigens has been the “gold standard” for the past two decades. Yet, the challenges remain: hand-picked antibody panels are primarily subjective, and the IHC analysis can identify primary sites in only 50-65% of patients with metastases and an even lower rate of 20-25% in CUP patients. In recent years, with the development of biotechnology, researchers have been able to detect the gene expression, miRNA, lncRNA, and methylation levels of tumor tissue cells, and obtain tissue-specific tumor characteristics through analysis. The molecular expression profiles of tumor cells in metastases are more similar to those in the primary tumor, but different from those in the metastatic site. This suggests that tumor origin can be traced based on the molecular expression profiles of metastatic tumor cells. At present, studies have constructed tumor traceability models, and achieved high accuracy by analyzing mRNA, miRNA, or lncRNA levels. Compared to the single-stranded RNAs, the double-stranded nature and the absence of a reactive 2′-hydroxyl group on the pentose ring make DNA more attractive for genetic testing. DNA methylation, the addition of a methyl group to the cytosine almost exclusively in the context of CpG dinucleotides, shows both cell- and tissue-specific patterns in the human genome, which can be used to determine the primary location of the tumor. Machine learning algorithms can discover patterns from a large amount of methylation data, and classify the tumor tissue origin accordingly. As a result, the machine learning algorithms are suitable for the methylation-based tumor tracing.

### SUMMARY

In order to overcome the low accuracy and effectiveness of IHC marker recognition in the prior part, the present disclosure designs a novel DNA methylation-based technology, including methylated adapters with inline barcodes and matched PCR amplification primers. The technology has higher accuracy and effectiveness.

To achieve the above objective, the present disclosure adopts the following technical solutions: the present disclosure provides a kit for identifying tumor tissue-of-origin, including a serial methylated adapter that have nucleotide sequences of A01-T, A01-B, A02-T, A02-B, A03-T, A03-B, A04-T, A04-B, A05-T, A05-B, A06-T, and A06-B.

Preferably, the kit for identifying tumor tissue-of-origin further includes PCR amplification primers and a PCR amplification reagent, where the PCR amplification primers include nucleotide sequences of R01-F, R01-R, R02-F, and R02-R.

Preferably, the kit for identifying tumor tissue-of-origin further includes a PCR amplification reagent, where the PCR amplification reagent includes a polymerase, dNTP, MgCl₂, and Tris-HCl.

Preferably, the kit for identifying tumor tissue-of-origin further includes a dephosphorylase and a 10× buffer selected from the group consisting of KAc, Tris-Ac, and Mg(Ac)₂. The buffer works well for multiple enzymatic reactions employed in this kit.

Preferably, the kit for identifying tumor tissue-of-origin further includes a dNTP mixture, where the dNTP mixture includes dATP, dCTP, and dGTP and excludes dTTP.

Preferably, the kit for identifying tumor tissue-of-origin further includes an end repair enzyme, a ligase, and ATP.

Preferably, the kit for identifying tumor tissue-of-origin further includes a negative control and a positive control, where the negative control is a healthy human blood leukocyte DNA, and the positive control is a cancer tissue sample DNA.

The present disclosure further provides a data analysis method for a kit for identifying tumor tissue-of-origin, including the following steps:
1) data preprocessing: conducting quality control on a raw off-machine data, and removing an adapter sequence and an inline barcode sequence of a raw data to obtain clean data;
2) alignment: allowing the clean data to be aligned to the human reference genome, and converting a resulting bam file generated by the alignment into an mHap file;
3) CpG methylation information statistics: extracting a methylation information of each CpG site;
4) quality control: removing a sample with less than 800,000 CpG sites and having a bisulfite conversion rate of less than 99%, an alignment rate of less than 50%, and a coverage of not less than 10×; and
5) analysis: analyzing a Beta value-based DNA methyaltion index, predicting all samples in sequence with a training set model, and outputting a probability value of each sample on 10 cancer types.

Preferably, the quality control is conducted on the raw off-machine data by a FastQC software component in step 1).

Preferably, the adapter sequence and the inline barcode sequence of the raw data are removed by Trim Galore software in step 1).

The beneficial effects of the present disclosure are: (1) The present disclosure is based on two methodological patents including “CN13604540A, Method for Quickly Constructing RRBS Sequencing Library Using Circulating Tumor DNA” and “CN13550013A, Method for Rapidly Constructing RRBS Sequencing Library Using Formalin-fixed Paraffin-Embedded Sample”. In the present disclosure, a kit is developed for qualitatively detecting a methylation profile of a human genome in formalin-fixed paraffin-embedded (FFPE) tumor tissues or blood samples of patients initially diagnosed as cancer by clinicians. The kit has higher accuracy and effectiveness. The present disclosure solves the current problem that the primary tumor of some tumor patients cannot be determined by IHC. (2) The present disclosure further improves the prediction accuracy of tumor primary site through a matching analysis system.

### BRIEF DESCRIPTION OF THE DRAWINGS

FIG. 1 shows a flowchart of the methylation detection of the present disclosure;

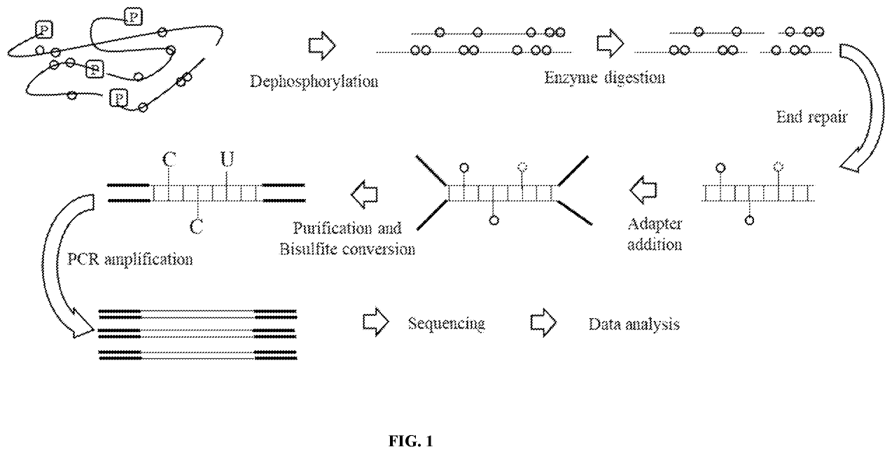

FIG. 2 shows a flowchart of constructing an analysis model of the present disclosure; and

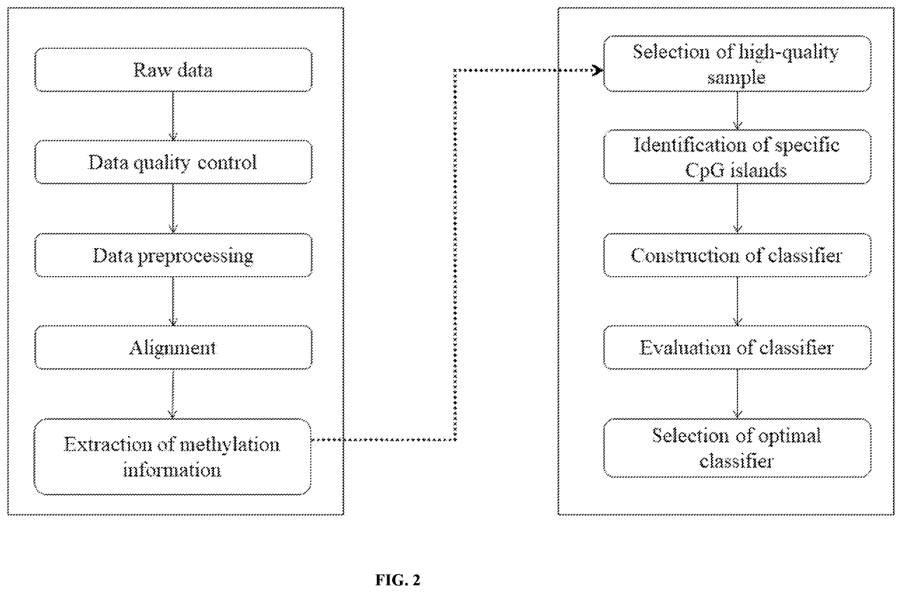

FIG. 3 shows a flowchart of the methylation analysis of the present disclosure.

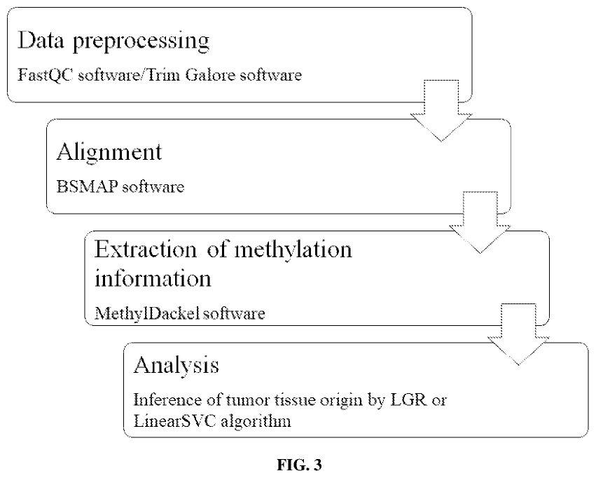

### DETAILED DESCRIPTION OF THE EMBODIMENTS

The following further describes the present disclosure in detail with reference to the accompanying drawings and examples.

In one example, the present disclosure provides a kit for identifying tumor tissue-of-origin, including an adapter, PCR amplification primers, PCR amplification reagents, a dephosphorylase, a 10× buffer, a dNTP mixture, an end repair enzyme, a ligase, ATP, a negative control, and a positive control, where the adapter includes nucleotide sequences of A01-T, A01-B, A02-T, A02-B, A03-T, A03-B, A04-T, A04-B, A05-T, A05-B, A06-T, and A06-B. The PCR amplification reagent includes a polymerase, dNTP, MgCl₂, and Tris-HCl. The 10× buffer is selected from the group consisting of KAc, Tris-Ac, and Mg(Ac)₂.

Adapter nucleotide sequences are specifically as follows:

| | |
|---|---|
| A01-T | mCTmCAmCGAmCGmCTmCTmCmCGTmCTAmC |
| | AAmCmC-S-T (SEQ ID NO: 1) |
| A01-B | Pho-GGTTGTAGATmCGGAAGAGmCAmCAmC |
| | GTmCTAAmC (SEQ ID NO: 2) |
| A02-T | mCTAmCAmCGmCGmCTmCTmCmCGATmCTAm |
| | CTmCAmC-S-T (SEQ ID NO: 3) |
| A02-B | Pho-GTGAGTAGATmCGGAAGAGmCAmCAmC |
| | GTmCTGAAmC (SEQ ID NO: 4) |
| A03-T | mCTAmCAmCGmCGmCTmCTmCmCGATmCTAG |
| | GATG-S-T (SEQ ID NO: 5) |
| A03-B | Pho-mCATmCmCTAGATmCGGAAGAGmCAmC |
| | AmCGTmCTGAAmC (SEQ ID NO: 6) |
| A04-T | mCTAmCAmCAmCGmCTmCTTmCmCGATmCTA |
| | TmCGAmC-S-T (SEQ ID NO: 7) |
| A04-B | Pho-GTmCGATAGATmCGGAAGAGmCAmCAm |
| | CGTmCTGAAmC (SEQ ID NO: 8) |
| A05-T | mCTAmCAmCGAmCGmCTmCTTmCmCGTmCTm |
| | CAAGAG-S-T (SEQ ID NO: 9) |
| A05-B | Pho-mCTmCTTGAGATmCGGAAGAGmCAmCA |
| | mCGTmCTGAAmC (SEQ ID NO: 10) |
| A06-T | mCTAmCAmCGAmCGmCTmCTTmCmCGATmCT |
| | mCATAmC-S-T (SEQ ID NO: 11) |
| A06-B | Pho-GTmCATGAGATmCGGAAGAGmCAmCAm |
| | CmCTGAAmC (SEQ ID NO: 12) |

The PCR amplification primers include nucleotide sequences of R01-F, R01-R, R02-F, and R02-R. The primer nucleotide sequences of the PCR amplification primers are specifically as follows:

| | |
|---|---|
| R01-F | AATGATACGGCGACCACCGCACTCTTTCCCTACACGACG |
| | CTCTTCCGATCT (SEQ ID NO: 13) |
| R01-R | CAAGCAGAAGACGGCATACGAGATTCGCCTTAGTGAGTT |
| | CAGACGTGTGCTCTTCCGATCT (SEQ ID NO: 14) |
| R02-F | AATGATACGGCGACCACCGATACACTCTTTCCCTACACG |
| | ACGCTCTTCCGATCT (SEQ ID NO: 15) |
| R02-R | CAAGCAGAAGACGGCATACGAGATCTAGTACGGTGTCAG |
| | ACGTGTGCTCTTCCGATCT (SEQ ID NO: 16) |

In another example, the present disclosure provides a data analysis method for a kit for identifying tumor tissue-of-origin, including the following steps:
1) data preprocessing: conducting quality control on a raw off-machine data, and removing an adapter sequence and an inline barcode sequence of a raw data to obtain clean data; the quality control is conducted on the raw off-machine data by a FastQC software component in step 1); the adapter sequence and the inline barcode sequence of the raw off-machine data are removed by Trim Galore software in step 1);
2) alignment: allowing the clean data to be aligned to the human reference genome, and converting a resulting bam file generated by the alignment into an mHap file;
3) CpG methylation information statistics: extracting a methylation information of each CpG site;
4) quality control: removing a sample with less than 800,000 CpG sites and having a bisulfite conversion rate of less than 99%, an alignment rate of less than 50%, and a coverage of not less than 10×; and
5) analysis: analyzing a Beta value-based methylation index, predicting all samples in sequence with a training set model, and outputting a probability value of each sample on 10 cancer types.

### Example 1: A Detection Process of this Product was as Follows

### 1) DNA Dephosphorylation Treatment:

A DNA dephosphorylation system included: 5 ng to 500 ng of DNA, 0.1 μL to 1.0 μL of dephosphorylase, 0.3 μL to 3.0 μL of buffer, and adding water to a total volume of 10 μL to 50 μL. The DNA dephosphorylation included: treatment at 37° C. for 5 min to 50 min, and treatment at 75° C. for 5 min to 30 min.

### 2) DNA Digestion:

A DNA digestion system included: 0.1 μL to 1.0 μL of endonuclease and 0.1 μL to 1.0 μL of buffer were added to the DNA dephosphorylation treatment system. DNA digestion included: treatment at 37° C. for 10 min to 100 min, and treatment at 70° C. for 5 min to 30 min.

### 3) End Repair of the Library:

An end repair system included: 0.1 μL to 1.0 μL of end repair enzyme, 0.1 μL to 1.0 μL of buffer, and 0.1 μL to 1.0 μL of dNTP mixture were added to the DNA digestion system. End repair reaction included: treatment at 30° C. for 10 min to 30 min, treatment at 37° C. for 10 min to 30 min, treatment at 70° C. for 5 min to 30 min.

### 4) Adapter Ligation:

An adapter ligation system included: 0.1 μL to 1.0 μL of ATP, 0.4 μL to 4.0 μL of ligase, 0.5 μL to 5.0 μL of buffer, and 0.3 μL to 3.0 μL of adapter were added to the library end repair system. An adapter ligation procedures included: treatment at 16° C. for 1 h to 4 h, treatment at 70° C. for 10 min to 30 min.

### 5) Nucleic Acid Purification:

The system was purified with a nucleic acid purification reagent, and 40 μL to 60 μL of a DNA solution was taken into a transformation reaction.

### 6) Bisulfite Conversion:

The purified nucleic acid was subjected to sodium bisulfite conversion with a conversion kit.

### 7) PCR Amplification:

A PCR amplification system included: 20 μL to 30 μL of transformed DNA, 10 μL to 25 μL of PCR amplification reagent, and 1 μL to 5 μL of PCR amplification primers.

### PCR Amplification Program (as Shown in the Table Below):

| | | |
|---|---|---|
| Temperature | Time | Number of cycles |

| | | | |
|---|---|---|---|
| 98° | C. | 45 sec | 1 |
| 98° | C. | 5 sec | 6 |
| 58° | C. | 5 sec | |
| 72° | C. | 10 sec | |
| 98° | C. | 5 sec | 14 |
| 65° | C. | 5 sec | |
| 72° | C. | 10 sec | |
| 72° | C. | 5 min | 1 |
| 4° | C. | hold | |

### 8) Library Purification:

The library was purified with a nucleic acid purification reagent, and 10 μL to 50 μL of the DNA solution was stored in a 1.5 mL EP tube.

### 9) Library Quality Control:

Quality control of library concentration: Qubit® dsDNA HS Assay Kit was used for quantifying a library concentration, and the library concentration should be greater than 2.5 ng/μL. Quality control of library fragment length: Agilent High Sensitivity DNA Kit was used for quality inspection of library fragments. If the size distribution of the library DNA was between 200 bp to 400 bp, the library was considered as qualified.

10) Convention of General Library into MGI Library (Illumina Platform Skipped this Step):

A universal library was converted into an MGI library with an MGIEasy Universal Library Conversion Kit.

### 11) Sequencing:

Sequencing was conducted with the matching sequencing reagents of a gene sequencer MGISEQ-2000 or the matching sequencing reagents of an Illumina sequencer.

A data analysis method for the kit for identifying tumor tissue-of-origin included:
1) Data preprocessing: quality control was conducted on a raw off-machine data with a FastQC software component, and an adapter sequence and an inline barcode sequence of a raw data were removed with Trim Galore software to obtain clean data.
2) Alignment: the clean data was aligned to the human reference genome (version hg19), and a resulting bam file generated by the alignment was converted into an mHap file.
3) CpG methylation information statistics: a methylation information of each CpG site was extracted with methylation data analysis software MethylDackel.
4) Quality control: to obtain high-quality samples, a sample with less than 800,000 CpG sites and having a bisulfite conversion rate of less than 99%, an alignment rate of less than 50%, and a coverage of not less than 10× was removed.
5) Feature selection: CpG islands that met the conditions for subsequent analysis were selected. A specific screening method was as follows: the coverage of CpG islands should be greater than 100×. Moreover, potential CpG islands that could be used as biomarkers by t-test were selected from the DNA methylation data set of 498 primary tumors and 57 tumor-adjacent normal tissues. The selected CpG islands should exhibit methylation levels in one cancer type that were significantly different (FDR≤0.01) from those in other cancer types. Moreover, the methylation level should be significantly higher or lower than 57 tumor-adjacent normal tissues (FDR≤0.01).

In this example, 4 different methods were used to characterize the methylation level of CpG islands. The 4 methods were: mean methylation (Beta value), Proportion of Discordant Reads (PDR), Cell Heterogeneity-Adjusted cLonal Methylation (CHALM), and Methylated Haplotype Load (MHL). Each method could obtain a set of CpG islands, which were used for subsequent classifier construction.
6) Classifier construction: 28 classifiers were constructed using 7 machine learning algorithms (adaboost, KNN, LGR, LinearSVC, NB, RF, and SVM) and the above 4 sets of CpG islands, and then a validation set was used to evaluate these classifiers. The main indicators of evaluation included Precision, Recall, F1 score, and Accuracy. The calculation formulas were as follows:

$$Recall = TP / ( TP + FN )$$

$$Precision = TP / ( TP + FP )$$

$$F 1 = 2 ( recall * precision ) / ( recall + precision )$$

$$Accuracy = ( TP + TN ) / ( TP + FP + TN + FN )$$

The predicted AUC of the classifier constructed based on the above method (the table below)

| Methods | Beta | CHALM | MHL | PDR |
|---|---|---|---|---|
| adaboost | 0.88 | 0.56 | 0.88 | 0.52 |
| KNN | 0.81 | 0.58 | 0.80 | 0.54 |
| LGR | 0.95 | 0.58 | 0.92 | 0.60 |
| LinearSVC | 0.95 | 0.54 | 0.92 | 0.57 |
| NB | 0.58 | 0.53 | 0.55 | 0.54 |
| RF | 0.88 | 0.57 | 0.86 | 0.58 |
| SVM | 0.90 | 0.61 | 0.85 | 0.59 |

In is example, the LGR and LinearSVC algorithms could also be used to analyze the Beta value-based methylation index, showing the maximum AUC.

### Example 2

The produced human genome methylation detection kit was tested, the clinical samples from different hospitals were detected, and the clinicopathologic diagnosis method was used as a comparative verification method.

Comparative verification method—the clinicopathologic diagnosis method is based on the current medical level and is the result of a comprehensive determination of information by professional doctors, where the information included blood and biochemical tests, urinalysis, fecal occult blood test, radiological examination of the suspected tumor primary, pathological immunohistochemistry, and medical history.

A testing method of this kit included the following steps:

Paraffin-embedded pathological tissues: a sample was collected from the lesion tissues to confirm that there were at least 30% tumor-related lesion tissues. Paraffin-embedded pathological tissues or section samples were confirmed to carry neoplastic lesion cells. The samples that had been stored for not more than 1 year were selected, and DNA extraction was conducted with a commercial kit using not less than 8 pieces of 5 μm sections or no less than 5 pieces of 10 μm sections.

An effective DNA concentration was determined by Qubit® dsDNA HS Assay Kit. If the DNA concentration was not less than 2 ng/μL, and a total amount of DNA was not less than 50 ng, it was determined to be qualified.

### 1) DNA Dephosphorylation Treatment:

A DNA dephosphorylation system included: 50 ng of DNA, 1.0 μL of dephosphorylase, 10 μL of buffer, and adding water to a total volume of 30 μL. DNA dephosphorylation included: treatment at 37° C. for 50 min, and treatment at 75° C. for 20 min.

### 2) DNA Digestion:

A DNA digestion system included: 1.0 μL of endonuclease and 0.2 μL of buffer were added to the DNA dephosphorylation treatment system. DNA digestion included: treatment at 37° C. for 90 min, and treatment at 70° C. for 10 min.

### 3) End Repair of the Library:

An end repair system included: 1.0 μL of end repair enzyme, 0.4 μL of buffer, and 0.8 μL of dNTP mixture were added to the DNA digestion system. End repair reaction included: treatment at 30° C. for 25 min, treatment at 37° C. for 25 min, treatment at 70° C. for 10 min.

### 4) Adapter Ligation:

An adapter ligation system included: 0.2 μL of ATP, 0.4 μL of ligase, 0.6 μL of buffer, and 3.0 μL of adapter were added to the library end repair system. An adapter ligation procedures included: treatment at 16° C. for 4 h, treatment at 70° C. for 15 min.

### 5) Nucleic Acid Purification:

The system was purified with a nucleic acid purification reagent, and 40 μL of a DNA solution was taken into a transformation reaction.

### 6) Bisulfite Conversion:

The purified nucleic acid was subjected to sodium bisulfite conversion with a conversion kit.

### 7) PCR Amplification:

A PCR amplification system included: 20 μL of transformed DNA, 25 μL of PCR amplification reagent, and 5 μL of PCR primers.

### PCR Amplification Program (as Shown in the Table Below):

| | | |
|---|---|---|
| Temperature | Time | Number of cycles |

| | | | |
|---|---|---|---|
| 98° | C. | 45 sec | 1 |
| 98° | C. | 5 sec | 6 |
| 58° | C. | 5 sec | |
| 72° | C. | 10 sec | |
| 98° | C. | 5 sec | 14 |
| 65° | C. | 5 sec | |
| 72° | C. | 10 sec | |
| 72° | C. | 5 min | 1 |
| 4° | C. | hold | |

### 8) Library Purification:

The library was purified with a nucleic acid purification reagent, and 35 μL of the DNA solution was stored in a 1.5 mL EP tube.

### 9) Library Quality Control:

Quality control of library concentration: Qubit® dsDNA HS Assay Kit was used for quality control of a library concentration, and the library concentration was greater than 2.5 ng/μL. Quality control of library fragment length: Agilent High Sensitivity DNA Kit was used for quality inspection of library fragments. If the size of the main band of the fragment was 200 bp to 400 bp, the library fragment was qualified.

10) Convention of General Library into MGI Library:

A universal library was converted into an MGI library with an MGIEasy Universal Library Conversion Kit.

### 11) Sequencing:

Sequencing was conducted with the matching sequencing reagents of a gene sequencer MGISEQ-2000.

### Example 3

The present disclosure further provided a data analysis method for a kit for identifying tumor tissue-of-origin, including the following steps:
1) Data preprocessing: quality control was conducted on a raw off-machine data with a FastQC software component, and an adapter sequence and an inline barcode sequence of a raw data were removed with Trim Galore software to obtain clean data.
2) Alignment: the clean data was aligned to the human reference genome (version hg19), and a resulting bam file generated by the alignment was converted into an mHap file.
3) CpG methylation information statistics: a methylation information of each CpG site was extracted with methylation data analysis software MethylDackel.
4) Quality control: a sample with less than 800,000 CpG sites and having a bisulfite conversion rate of less than 99%, an alignment rate of less than 50%, and a coverage of not less than 10× was removed.
5) Analysis: a Beta value-based methylation index was analyzed with LGR and LinearSVC algorithms, all samples were predicted in sequence with a training set model, and a probability value of each sample on 10 cancer types was output.
6) The consistency analysis results were shown in summary Table 1. The analysis showed the prediction results of the human genome methylation detection kit (combined with a probe-anchored polymerization sequencing method) on the primary site of cancer (according to the probability value, a first-ranked result was selected to be compared with a pathological diagnosis result), with an overall accordance rate of 78.75%. The above results had high total accordance rate and consistency with the clinical diagnosis results, thus preliminarily verifying that the kit showed high accuracy and effectiveness in detecting the primary site of cancer.

TABLE 1
Consistency analysis results (compared with
results ranked first in probability value)

| | | | Samples with | |
|---|---|---|---|---|
| | | Sample | accurate | Accordance |
| SN | Cancer name | size | prediction | rate |
| 1 | Breast cancer | 27 | 24 | 88.89% |
| 2 | Lung cancer group | 27 | 23 | 85.19% |
| 3 | Gastric cancer | 30 | 20 | 66.67% |
| 4 | Intestinal cancer | 31 | 30 | 96.77% |
| 5 | Head and neck cancer | 22 | 15 | 68.18% |
| 6 | Thyroid cancer | 25 | 23 | 92.00% |
| 7 | Cervical cancer | 16 | 9 | 56.25% |
| 8 | ovarian cancer | 26 | 21 | 80.77% |
| 9 | Liver cancer | 23 | 18 | 78.26% |
| 10 | Esophagus cancer | 13 | 6 | 46.15% |
| Total | Total number of samples | 240 | 189 | 78.75% |

Considering that some cancers such as cervical cancer only occur in women, and when a difference between the first and second predicted probability values is small, it is possible that a corresponding part of the predicted probability value ranked second is the real primary cancer lesion. Therefore, the top two results of the predicted probability values were analyzed, and one of the top two consistent with the clinical diagnosis result was regarded to be consistent. The results were shown in the table. The accordance rate of cancer types including gastric cancer, head and neck cancer, cervical cancer, and esophagus cancer had been significantly improved, with an overall accuracy increased to 88.33% (Table 2).

TABLE 2
Consistency analysis results (compared with
results ranked TOP2 in probability value)

| | | | TOP2 | |
|---|---|---|---|---|
| | | Sample | prediction | Accuracy |
| SN | Cancer name | size | samples | rate |
| 1 | Breast cancer | 27 | 25 | 92.59% |
| 2 | Lung cancer group | 27 | 26 | 96.30% |
| 3 | Gastric cancer | 30 | 22 | 73.33% |
| 4 | Intestinal cancer | 31 | 30 | 96.77% |
| 5 | Head and neck cancer | 22 | 20 | 90.91% |
| 6 | Thyroid cancer | 25 | 25 | 100.00% |
| 7 | Cervical cancer | 16 | 12 | 75.00% |
| 8 | ovarian cancer | 26 | 22 | 84.62% |
| 9 | Liver cancer | 23 | 20 | 86.96% |
| 10 | Esophagus cancer | 13 | 10 | 76.92% |
| Total | Total number of | 240 | 212 | 88.33% |
| | samples | | | |

Among the 240 samples tested in this study, 89 carcinomas were known to be poorly or moderately differentiated. The results were shown in Table 3. Calculated according to the highest predicted probability value (TOP1), the accuracy could reach 75.28%. The accuracy was increased to 85.39% by including the top two predicted probability values (TOP2) in the calculation. This indicated that the ability of this product to determine the primary site of poorly differentiated cancer was better than that of immunohistochemistry reported by the current technical level.

TABLE 3
Consistency analysis results of moderately
and poorly differentiated cancer tissues

| | | Sam- | TOP1 | | TOP2 | |
|---|---|---|---|---|---|---|
| | Cancer | ple | prediction | Accuracy | prediction | Accuracy |
| SN | name | size | samples | rate | samples | rate |
| 1 | Breast | N/A | N/A | N/A | N/A | N/A |
| | cancer | | | | | |
| 2 | Lung | 14 | 11 | 78.57% | 13 | 92.86% |
| | cancer | | | | | |
| | group | | | | | |
| 3 | Gastric | 21 | 15 | 71.43% | 16 | 76.19% |
| | cancer | | | | | |
| 4 | Intestinal | 17 | 16 | 94.12% | 16 | 94.12% |
| | cancer | | | | | |
| 5 | Head and | 12 | 8 | 66.67% | 11 | 91.67% |
| | neck | | | | | |
| | cancer | | | | | |
| 6 | Thyroid | N/A | N/A | N/A | N/A | N/A |
| | cancer | | | | | |
| 7 | Cervical | 6 | 3 | 50.00% | 3 | 50.00% |
| | cancer | | | | | |
| 8 | ovarian | 9 | 9 | 100.00% | 9 | 100.00% |
| | cancer | | | | | |
| 9 | Liver | 5 | 4 | 80.00% | 4 | 80.00% |
| | cancer | | | | | |
| 10 | Esophagus | 5 | 1 | 20.00% | 4 | 80.00% |
| | cancer | | | | | |
| Total | | 89 | 67 | 75.28% | 76 | 85.39% |

In general, the determination result of this product had high total accordance rate and consistency with those of the clinical diagnosis results. This preliminarily verified that the human genome methylation detection kit had a clinical value for the detection of the primary site of cancer, and showed high accuracy and effectiveness.

The foregoing is merely a preferable example of the present disclosure without limitation on the scope of the present disclosure. Any equivalent structure change made by using the description and the accompanying drawings of the present disclosure, or direct or indirect application thereof in other related technical fields, shall still fall in the protection scope of the patent of the present disclosure.

What is claimed is:

1. A kit for identifying tumor tissue-of-origin, comprising an adapter, wherein the adapter comprises nucleotide sequences of A01-T, A01-B, A02-T, A02-B, A03-T, A03-B, A04-T, A04-B, A05-T, A05-B, A06-T, and A06-B.

2. The kit for identifying tumor tissue-of-origin according to claim 1, further comprising PCR amplification primers and a PCR amplification reagent, wherein the PCR amplification primers comprise nucleotide sequences of R01-F, R01-R, R02-F, and R02-R.

3. The kit for identifying tumor tissue-of-origin according to claim 1, further comprising a PCR amplification reagent, wherein the PCR amplification reagent comprises a polymerase, dNTP, MgCl₂, and Tris-HCl.

4. The kit for identifying tumor tissue-of-origin according to claim 1, further comprising a dephosphorylase and a 10× buffer, wherein the 10× buffer is selected from the group consisting of KAc, Tris-Ac, and Mg(Ac)₂.

5. The kit for identifying tumor tissue-of-origin according to claim 1, further comprising a dNTP mixture, wherein the dNTP mixture comprises dATP, dCTP, and dGTP.

6. The kit for identifying tumor tissue-of-origin according to claim 1, further comprising an end repair enzyme, a ligase, and ATP.

7. The kit for identifying tumor tissue-of-origin according to claim 2, further comprising an end repair enzyme, a ligase, and ATP.

8. The kit for identifying tumor tissue-of-origin according to claim 3, further comprising an end repair enzyme, a ligase, and ATP.

9. The kit for identifying tumor tissue-of-origin according to claim 4, further comprising an end repair enzyme, a ligase, and ATP.

10. The kit for identifying tumor tissue-of-origin according to claim 5, further comprising an end repair enzyme, a ligase, and ATP.

11. The kit for identifying tumor tissue-of-origin according to claim 1, further comprising a negative control and a positive control, wherein the negative control is a healthy human blood leukocyte DNA, and the positive control is a cancer tissue sample DNA.

12. The kit for identifying tumor tissue-of-origin according to claim 2, further comprising a negative control and a positive control, wherein the negative control is a healthy human blood leukocyte DNA, and the positive control is a cancer tissue sample DNA.

13. The kit for identifying tumor tissue-of-origin according to claim 3, further comprising a negative control and a positive control, wherein the negative control is a healthy human blood leukocyte DNA, and the positive control is a cancer tissue sample DNA.

14. The kit for identifying tumor tissue-of-origin according to claim 4, further comprising a negative control and a positive control, wherein the negative control is a healthy human blood leukocyte DNA, and the positive control is a cancer tissue sample DNA.

15. The kit for identifying tumor tissue-of-origin according to claim 5, further comprising a negative control and a positive control, wherein the negative control is a healthy human blood leukocyte DNA, and the positive control is a cancer tissue sample DNA.

16. A data analysis method for a kit for identifying tumor tissue-of-origin, comprising the following steps:
1) data preprocessing: conducting quality control on a raw off-machine data, and removing an adapter sequence and an inline barcode sequence of a raw data to obtain clean data;
2) alignment: allowing the clean data aligned to the human reference genome, and converting a resulting bam file generated by the alignment into an mHap file;
3) CpG methylation information statistics: extracting a methylation information of each CpG site;
4) quality control: removing a sample with less than 800,000 CpG sites and having a bisulfite conversion rate of less than 99%, an alignment rate of less than 50%, and a coverage of not less than 10×; and
5) analysis: analyzing a Beta value-based methylation index, predicting all samples in sequence with a training set model, and outputting a probability value of each sample on 10 cancer types.

17. The data analysis method for a kit for identifying tumor tissue-of-origin according to claim 16, wherein the quality control is conducted on the raw off-machine data by a FastQC software component in step 1).

18. The data analysis method for a kit for identifying tumor tissue-of-origin according to claim 17, wherein the adapter sequence and the inline barcode sequence of the raw data are removed by Trim Galore software in step 1).
